(19) United States
(12) Patent Application Publication
(10) Pub. No.: US 2021/0095333 A1
(43) Pub. Date: Apr. 1, 2021

(54) QUANTIFICATION OF MOLECULES USING NUCLEIC ACID STRAND DISPLACEMENT DETECTION

(71) Applicant: X Development LLC, Mountain View, CA (US)

(72) Inventors: Anupama Thubagere Jagadeesh, Palo Alto, CA (US); Ashwin Gopinath, Palo Alto, CA (US)

(21) Appl. No.: 17/039,956

(22) Filed: Sep. 30, 2020

Related U.S. Application Data
(60) Provisional application No. 62/908,124, filed on Sep. 30, 2019.

(51) Int. Cl. C12Q 1/6834

(52) U.S. Cl. CPC C12Q 1/6834

(57) ABSTRACT

Methods of detecting and quantifying concentrations of a target molecule in a sample include determining the concentration of a displaced oligonucleotide strand.

### CROSS-REFERENCE TO RELATED APPLICATIONS

This application is a claims priority to Provisional Application No. 62/908,124, entitled “QUANTIFICATION OF MOLECULES USING NUCLEIC ACID STRAND DISPLACEMENT DETECTION,” filed on Sep. 30, 2019, the entire contents of which are incorporated herein by reference.

### TECHNICAL FIELD

Provided herein are methods and compositions for quantifying proteins, peptides, and small molecules.

### BACKGROUND

Over the last few decades, various detection methodologies have been developed based on identification of specific complex formation, including direct or indirect strategies that detect and/or amplify signals related to primary or secondary binding events, where signals could be optical (e.g., spectroscopic, colorimetric or fluorescent) or electrical (e.g., impedance, capacitance, inductance or current). Specific application areas of such platforms include: environmental assessment, food safety, medical diagnosis, and detection of chemical, biological and/or radiological warfare agents.

While the approaches described above have been very successful, they generally cannot be applied directly in situations for which the targets are at a very small concentration.

### SUMMARY

This disclosure is based, in part, on the surprising discovery that the presence of a target can be detected and quantified using strand displacement molecules.

Provided herein are methods and compositions to quantitatively convert the presence of a target, in a solution, into a nucleic acid of desired sequence such that low concentrations of the target can be detected. The system generally involves a nucleic acid strand displacement process in which a partially double stranded DNA, immobilized on a micro-particle, reacts simultaneously with a target and a helper DNA strand to release a DNA sequence of desired sequence into the solution. In this way, the concentration of the target can be quantified by quantifying the concentration of DNA in solution using a multitude of available high-throughput technologies.

Thus, in a first aspect, provided herein are methods for detecting a target in a sample. The methods include:

(i) contacting the sample with a detector molecule,

wherein the detector molecule includes a first oligonucleotide and a second oligonucleotide, wherein the first oligonucleotide in order from 5′ to 3′ or 5′ to 3′ includes a spacer region, a displacement region, a toehold region, and a detection region and the second oligonucleotide includes complementary regions to the displacement and toehold regions of the first oligonucleotide, with a third oligonucleotide including regions complementary to the displacement and toehold regions in the first oligonucleotide, under conditions that allow binding of the third oligonucleotide to the first oligonucleotide at both the displacement and toehold regions of the first oligonucleotide, thereby displacing the second oligonucleotide; and

(ii) detecting the second oligonucleotide displaced from the first oligonucleotide.

In some embodiments, the first, second, and third oligonucleotides include DNA, RNA, non-natural nucleic acids, or a combination thereof. In some embodiments, the first, second, and third oligonucleotides include DNA.

In some embodiments, detecting includes determining the concentration of the displaced second oligonucleotide by PCR or sequencing.

In some embodiments, the detection region in the first oligonucleotide binds at least one target.

In some embodiments, the first oligonucleotide is further fixed to a substrate at one end of the oligonucleotide. In some embodiments, the substrate is a bead or planar substrate.

In some embodiments, the method further includes removing the detector molecule and contacting the sample with a second detector molecule against a second target.

In some embodiments, the second oligonucleotide is further fixed to a substrate at one end of the oligonucleotide. In some embodiments, the second oligonucleotide is fixed to the substrate that is a bead or planar substrate.

In some embodiments, the target is a polypeptide or protein, or a combination thereof. In some embodiments, the target is complexed with a polypeptide or polynucleotide.

In some embodiments, the sample is a biological sample, e.g., a blood sample, a urine sample, a biopsy sample, or a saliva sample.

In some embodiments, the method further includes the target binds to the detector region.

The methods can also be used for identifying or producing a detector molecule for a target. For example, the methods can include:

contacting a sample with the detector molecule, wherein the sample includes the target,

wherein the detector molecule includes a first oligonucleotide and a second oligonucleotide, wherein the first oligonucleotide in order from 5′ to 3′ or 5′ to 3′ includes a first primer, a displacement region, a toehold region, a detection region, and a second primer and the second oligonucleotide includes complementary regions to the displacement and toehold regions of the first oligonucleotide and a barcode, with a third oligonucleotide including regions complementary to the displacement and toehold regions in the first oligonucleotide, under conditions that allow binding of the third oligonucleotide to the first oligonucleotide at both the displacement and toehold regions of the first oligonucleotide, thereby displacing the second oligonucleotide; and

determining the sequence of the detection region in the first oligonucleotide.

In some embodiments, the sequence of the detection region is random.

In some embodiments, the first, second, and third oligonucleotides include DNA, RNA, non-natural nucleic acids, or a combination thereof.

In some embodiments, determining the random sequence of the detection region of the first oligonucleotide is determined by PCR or next generation sequencing.

In some embodiments, the detection region in the first oligonucleotide binds at least one target. In some embodiments, the target is a polypeptide or protein, or a combination thereof. In some embodiments, the target is complexed with a polypeptide or a polynucleotide.

In some embodiments, the second oligonucleotide is further fixed to a substrate at one end of the oligonucleotide. In some embodiments, the substrate is a bead or planar substrate.

In some embodiments, the sample is a biological sample, e.g., a blood sample, a urine sample, a biopsy sample, or a sample.

Another aspect features a composition for detecting a target in a sample, the composition includes:

a detector molecule, wherein the detector molecule comprises a first oligonucleotide and a second oligonucleotide, wherein the first oligonucleotide in order from 5′ to 3′ or 5′ to 3′ includes a spacer region, a displacement region, a toehold region, and a detection region and the second oligonucleotide includes complementary regions to the displacement and toehold regions of the first oligonucleotide.

In some embodiments, the detector molecule comprises DNA, RNA, non-natural nucleic acids, or a combination thereof.

Another aspect features methods for detecting a biomarker in a subject. The method includes:

contacting a sample from the subject with a detector molecule,

wherein the sample comprises the biomarker,

wherein the detector molecule includes a first oligonucleotide and a second oligonucleotide, wherein the first oligonucleotide in order from 5′ to 3′ or 5′ to 3′ includes a spacer region, a displacement region, a toehold region, and a detection region and the second oligonucleotide includes complementary regions to the displacement and toehold regions of the first oligonucleotide, with a third oligonucleotide including regions complementary to the displacement and toehold regions in the first oligonucleotide, under conditions that allow binding of the third oligonucleotide to the first oligonucleotide at both the displacement and toehold regions of the first oligonucleotide, thereby displacing the second oligonucleotide;

detecting the second oligonucleotide displaced from the first oligonucleotide; and

determining the level of the biomarker in the sample compared to the level of the biomarker in a reference sample.

In some embodiments, the biomarker is selected from a group including prostate specific antigen, glucose, alpha-feto protein, monoclonal immunoglobulins, and pancreatic prohormone.

In some embodiments, wherein the sample is a biological sample, e.g., a blood sample, a urine sample, a biopsy sample, or a saliva sample.

In some embodiments, the reference sample is from a healthy subject.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety for any and all purposes. In case of conflict, the present specification, including definitions, will control.

Other features and advantages of the invention will be apparent from the following detailed description and figures, and from the claims.

### BRIEF DESCRIPTION OF DRAWINGS

FIG. 1A is a representation of strand displacement, e.g., a toehold mediated branch migration, is shown where one detector molecule (AB) interacts with another molecule (D), in the presence of a target (C), to result in a molecule (A) and a detectable reporting molecular construct (BD).

FIG. 1B is an illustration of a representative detector molecule. A first oligonucleotide contains a spacer, displacement, toehold, and detection region. A second oligonucleotide contains a barcode and complementary regions to the displacement and detection regions of the first oligonucleotide.

FIG. 1C is an illustration of a representative target, e.g., an oligonucleotide (target oligonucleotide) with complementary regions to the toehold and detection regions in the first oligonucleotide of the detector molecule or a polynucleotide, protein, peptide, small molecule, or polynucleotide/protein complex.

FIG. 1D is an illustration of a representative single stranded oligonucleotide (helper strand) with complementary regions to the displacement and toehold regions of the first oligonucleotide in the detector molecule that displaces the second oligonucleotide from the detector molecule.

FIG. 2A is a representation of a strand displacement process, e.g., toehold mediated branch migration strand displacement.

FIG. 2B is an illustration of a representative strand displacement process where the target is an oligonucleotide (molecule C in FIG. 1) with complementary regions to the toehold and detection regions in the first oligonucleotide in the detector molecule.

FIG. 2C is an illustration of a representative strand displacement process where the target is a polynucleotide, protein, peptide, small molecule, or polynucleotide/protein complex (molecule C in FIG. 1).

FIG. 3A is an illustration of representative strand displacement processes to identify detection regions for quantifying a target, e.g., polynucleotide, protein, peptide, small molecule, or polynucleotide/protein complex. The detector molecule is attached to a bead. The detection region of the detector molecule is a random sequence and will be sequenced and identified.

FIG. 3B is an illustration of a representative strand displacement process where the detector molecule is attached to a bead allowing for removable of the first oligonucleotide of the detector molecule with the target bound.

Like symbols in different figures indicate like elements.

### DETAILED DESCRIPTION

As used herein, the recited terms have the following meanings. All other terms and phrases used in the specification have their ordinary meanings as one skilled in the art would understand.

References in the specification to “one embodiment”, “an embodiment”, etc., indicate that the embodiment described may include a particular aspect, feature, structure, or characteristic, but not every embodiment necessarily includes that aspect, feature, structure, or characteristic. Moreover, such phrases may, but do not necessarily, refer to the same embodiment referred to in other portions of the specification. Further, when a particular aspect, feature, structure, or characteristic is described in connection with an embodiment, it is within the knowledge of one skilled in the art to affect or connect such aspect, feature, structure, or characteristic with other embodiments, whether or not explicitly described.

The singular forms “a”, “an”, and “the” include plural reference unless the context clearly dictates otherwise. Thus, for example, a reference to “a molecule” includes a plurality of such molecules.

As will also be understood by one skilled in the art, all language such as “up to”, “at least”, “greater than”, “less than”, “more than”, “or more”, and the like, include the number recited and such terms refer to ranges that can be subsequently broken down into sub-ranges. Specific values recited for ranges are for illustration only and they do not exclude other defined values or other values within defined ranges.

As used herein, the terms “nucleic acid”, “polynucleotide”, and “oligonucleotide” refer to deoxyribonucleotides or ribonucleotides and polymers thereof in either single- or double stranded form, composed of monomers (nucleotides) containing a sugar, phosphate and a base that is either a purine or pyrimidine. Unless specifically limited, the term encompasses nucleic acids containing known artificial nucleotides or analogs of natural nucleotides which have similar binding properties as the reference nucleic acid.

As used herein, the term “complementary” refers to a nucleic acid comprising a sequence of consecutive nucleobases capable of hybridizing to another nucleic acid strand even if less than all the nucleobases do not base pair with a counterpart nucleobase. In certain embodiments, a “complementary” nucleic acid comprises a sequence in which at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, and any range therein, of the nucleobase sequence is capable of base-pairing with another nucleic acid sequence.

As used herein, “biological sample” refers to any sample derived from a human, animal, plant, bacteria, fungus, virus, or yeast cell, including but not limited to tissue, blood, bodily fluids, serum, sputum, mucus, bone marrow, stem cells, lymph fluid, secretions, and the like.

A major challenge in the detection and quantification of a target in a sample is quantifying low concentrations and multiplexed molecules. Assays for detecting the presence of a target or determining the concentration of that target in a solution often entail binding a detection molecule to the target and an amplification procedure. For instance, standard amplification processes include enzyme-linked immunosorbent assays (ELISA) for amplifying the signal in antibody-based assays, as well as the polymerase chain reaction (PCR) for amplifying target DNA strands in DNA-based assays. Other hybrid amplification techniques also exist enabling protein targets to produce DNA signals, for example immunoPCR (see Sano, T.; Smith, C. L.; Cantor, C. R. Science 1992, 258, 120-122). Unfortunately, immunoPCR is a complex assay and can be prone to false positive signal generation (Niemeyer, C. M.; Adler, M.; Wacker, R. Trends in Biotechnology 2005, 23, 208-216).

These assays, due to the presence of the amplification step, are impracticable to multiplex for multiple targets or complexed targets. Further, approaches to use such assays for quantification often involve a series of dilution steps which lead to a large amount of sample. Thus there is a large need for a modular, multiplexable method to quantitatively measure the presence of targets in small sample volumes.

As described herein, methods and compositions relate to an enzyme-free technique determining the concentration of targets in a fluid sample. The first method is used to quantify a target in a solution. Detector molecule have a polynucleotide construct that is tailored to release a single DNA strand of user-designed sequence, upon interacting with a target. The released DNA strand is quantified using standard genomic techniques like PCR, digital PCR, or next generation sequencing. The second method identifies polynucleotide constructs that result in the release of a unique DNA strand upon interaction with a target and helper DNA strand.

Toehold mediated branch migration strand displacement results in displacement of one strand in a double stranded/single stranded oligonucleotide molecule. The detector molecule includes a spacer, displacement, toehold and detection regions on one oligonucleotide and a second oligonucleotide includes a barcode and complementary regions to the displacement region and toehold region of the first oligonucleotide.

The detector region of the detector molecule is designed to specifically bind to the target, (e.g., polynucleotide, protein, peptide, small molecule, or polynucleotide/protein complex) and partially denature at the toehold region upon binding of the target. The toehold region is designed to denature upon binding of the target. The displacement region, on both the first and second oligonucleotides, forms a double strand DNA region. In some embodiments, the spacer region is a primer used for next generation sequencing. In some embodiments, a substrate is added to the end at the spacer region. In some embodiments the barcode region is unique to each detector molecule including a unique detection region. Next generation sequencing can be used to sequence the barcode and identify the unique detector molecule. Further, a helper strand in the solution is designed to interact with the partially denatured toehold region resulting in complete denaturation of the detector molecule.

Denaturation only occurs in the presence of the target (e.g., polynucleotide, protein, peptide, small molecule, or polynucleotide/protein complex). Toehold mediated branch migration strand displacement occurs after the target interacts with the detector molecule, the second oligonucleotide is released from the detector molecule and can be used as a marker for the quantification of the target.

Furthermore, methods can be designed to search for the ideal polynucleotide sequence that can act as the “detector region” in a detector molecule for a given target.

### Oligonucleotides

The present disclosure provides oligonucleotides that may contain single stranded polynucleotide strands. The oligonucleotides may contain DNA, RNA, DNA/RNA, non-natural nucleic acid, or combinations thereof.

In some embodiments, the detector molecule includes a first oligonucleotide that has a spacer, displacement, toehold, and detection region, and further includes a second oligonucleotide that has a complementary sequence to the toehold and displacement regions of the first oligonucleotide. The second oligonucleotide further includes a barcode.

In some embodiments, the spacer region will not include G or C nucleotides. The spacer region can include A, T, and U nucleotides and non-natural analogs of A, T, and U nucleotides. In some embodiments, the displacement region will be longer than the toehold region. In some embodiments, the detector region will be longer than the toehold region. In some embodiments, the displacement region is at least 15 nucleotides. In some embodiments, the displacement region is not palindromic to the toehold region. The displacement region does not hybridize to the toehold region.

As used herein, the term “barcode” refers to a label, or identifier, that identifies a sample. A barcode can be oligonucleotides that may contain DNA, RNA, DNA and RNA, non-natural nucleic acid, or a combination thereof. Barcodes can allow for identification and or quantification of individual samples. In some embodiments, an oligonucleotide can include one or more barcodes.

### Targets

As used herein, the terms “target” and “biomarkers” refer to a molecule that is detected or quantified by the presented methods and compositions. In some embodiments, the target is a polynucleotide, protein, peptide, small molecule, or polynucleotide/protein complex.

As used herein, “small molecules” refers to small organic or inorganic molecules of molecular weight below about 3,000 Daltons. In general, small molecules useful for the techniques disclosed herein have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

In some embodiments, the present methods and compositions can detect or quantify target or biomarker in a biological sample. Non-limiting examples of targets and biomarkers include albumin, alpha-feto protein, Beta-2 microglobulin, BCR/ABL1, cancer antigen 15-3, cancer antigen 19-9, cancer antigen 125, calcitonin, carcino-embryonic antigen, chromogranin A, des-gamma-carboxy prothrombin, fibrin, fibrinogen, gastrin, glucose, human chorionic gonadotropin, JAK2, lactate dehydrogenase, monoclonal immunoglobulins, prostate specific antigen, soluble mesothelin-related peptides, T-cell receptor, thyroglobulin. Further examples include, clusterin, α-1-microglobulin, IL-1ra, IL-6, IL-10, TNF-α, IL-13, ApoA1, transthyretin, IL-4, IL-2, IFN-γ, nucleosome assembly protein 2, PDGF, complement component C2, complement component C3, complement factor-I, α-1-microglobulin, serum amyloid-P, complement component C4a, complement component C8, α-1-antitrypsin, pancreatic prohormone, granulocyte colony-stimulating factor, insulin-like growth factor-binding protein 2, complement component C6, inter-alpha-trypsin inhibitor heavy chain H4, and C—C motif chemokine 18, serum amyloid A-1 protein, complement component C9, mannose-binding protein C, serum amyloid P-component, α2-antiplasmin, CHK1 (Serine/threonine-protein kinase Chk1), interleukin-17A, eukaryotic translation initiation factor 5A-1, hemopexin, CDCl37 (C—C motif chemokine 19), and complement factor H-related protein 5.

### Detection and Quantification

Detection and quantification of a target can be analyzed by digital droplet PCR or next generation sequencing. The barcode can be determined to identify the sample and target that is detected and quantified.

In some embodiments, optical readout detecting and quantifying the target can be analyzed when an anisotropic gold rod is attached to the top origami, then when the top origami binds the origami on the surface (when a target binds), the gold rod will go from freely rotating to fixed in a particular orientation. (Schickinger et al., “Tethered multifluorophore motion reveals equilibrium transition kinetics of single DNA double helices,” PNAS 115.32 (2018): E7512-E7521; Visser et al., “Continuous biomarker monitoring by particle mobility sensing with single molecule resolution,” Nature Communications 9.1 (2018): 2541; and WO2019059961A1). This change in rotational diffusion of the gold rod will be easily detected in a regular epifluorescence microscope by examining light in two different polarizations and calculating the ratio.

### Methods of Use

The methods and compositions described herein can be used to detect and quantify a single target in a sample. The detection region in the detector molecule can have affinity to a single target. In certain embodiments, the detector region can have affinity to a target when said target is complexed with another molecule, e.g., protein, DNA, RNA, and small molecule. For example, the detector region can have affinity to a surface on the target when the target is complexed with another molecule and only bind to the surface of the target. Alternatively, the detector region can have affinity to a surface on the target and a molecule that the target is complexed with.

In some embodiments, the methods and compositions described herein can be used to detect and quantify multiple targets in a sample. The detector molecule can have affinity to a conserved region on the surface of a family of target, e.g., conserved regions of a family of antibodies. Detection and quantification of the conserved region would represent the full family of targets and not just a single unique target.

In some embodiments, the methods and compositions described herein can be used to detect and quantify multiple targets without conserved regions in a sample. Multiple detector molecules, each having a unique bead or planar substrate, can detect multiple targets in a sample.

In some embodiments, the methods and compositions described herein can be used to detect and quantify a target in a sample obtained from a subject. The level of target determined in the sample can be compared to a reference level.

In some embodiments, the methods and compositions described herein can be used in a solution at a pH between 5.0-8.0. In certain embodiments, the solution has a pH between 5.5-8.0, between 6.0-8.0, between 6.5-8.0, between 7.0-8.0, and between 7.5-8.0. See Belleperche et al., Pharmaceuticals, 2018 11:80.

In some embodiments, the methods and compositions described herein can be used in a solution containing NaCl or another suitable salt at a concentration between 1 mM to 100 mM. In certain embodiments, the solution has a salt concentration between, 10 mM to 100 mM, between 20 mM to 100 mM, 30 mM to 100 mM, between 40 mM to 100 mM, between 50 mM to 100 mM, between 60 mM to 100 mM, between 70 mM to 100 mM, between 80 mM to 100 mM, between 90 mM to 100 mM, between 10 mM to 90 mM, between 20 mM to 90 mM, 30 mM to 90 mM, between 40 mM to 90 mM, between 50 mM to 90 mM, between 60 mM to 90 mM, between 70 mM to 90 mM, between 80 mM to 90 mM, between 10 mM to 80 mM, between 20 mM to 80 mM, 30 mM to 80 mM, between 40 mM to 80 mM, between 50 mM to 80 mM, between 60 mM to 80 mM, between 70 mM to 80 mM, between 10 mM to 70 mM, between 20 mM to 70 mM, 30 mM to 70 mM, between 40 mM to 70 mM, between 50 mM to 70 mM, between 60 mM to 70 mM, between 10 mM to 60 mM, between 20 mM to 60 mM, 30 mM to 60 mM, between 40 mM to 60 mM, between 50 mM to 60 mM, between 10 mM to 50 mM, between 20 mM to 50 mM, 30 mM to 50 mM, between 40 mM to 50 mM, between 10 mM to 40 mM, between 20 mM to 40 mM, 30 mM to 40 mM, between 10 mM to 30 mM, and between 20 mM to 30 mM.

In some embodiments, the methods and compositions described herein can be used in a suitable buffer, e.g., bis-tris, phosphate, MES, PBS (10 mmol/L sodium phosphate, 150 mmol/L NaCl), Tris buffer, and HEPES (4-(2-hydroxyethyl)-1-piperazineethanesulfonic acid). A suitable buffer can be adjusted to the suitable pH and salt concentration.

In some embodiments, the methods and compositions described herein can include a surfactant, from about 0.05% to about 0.1% by volume or by weight or the surfactant. The surfactant can be selected from the group consisting of non-ionic, cationic, anionic, and zwitterionic surfactants. In some embodiments, the surfactant is non-ionic, and can include, for example, polysorbate 20.

As used herein, a “reference sample” refers to a sample from a healthy subject, a subject not exhibiting symptoms of a disease, a subject exhibiting symptoms of a disease, and a collection of subjects that are healthy, not exhibiting symptoms of a disease, or exhibiting symptoms of a disease.

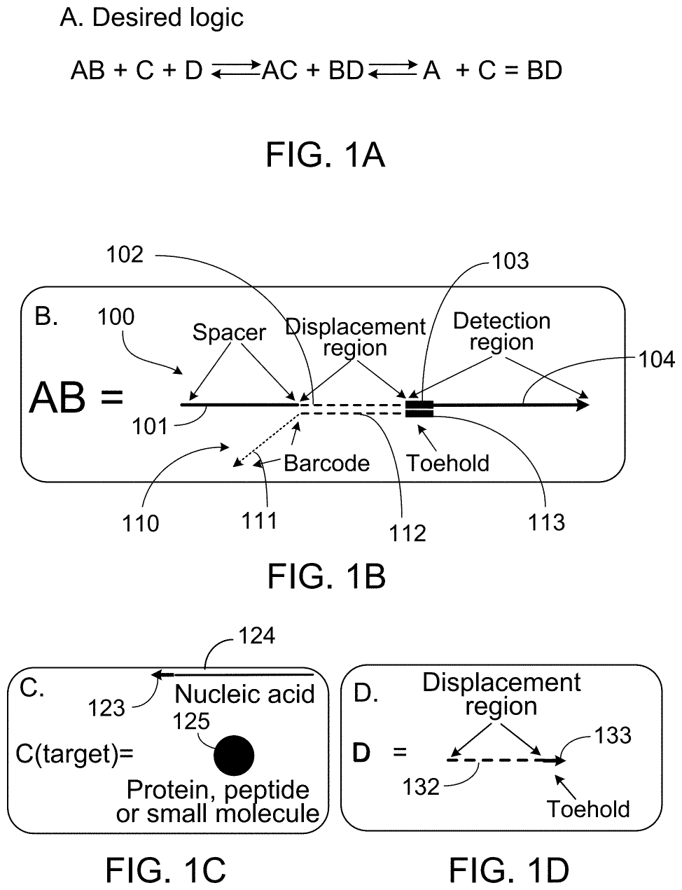

A formulaic representation of strand displacement is shown in FIG. 1A. Here, a toehold mediated branch migration is shown where one detector molecule (AB) interacts with another molecule (D), in the presence of a target (C), to result in a molecule (A) and a detectable reporting molecular construct (BD).

FIG. 1B is an illustration of a representative detector molecule. A first oligonucleotide 100 contains a spacer 101, displacement 102, toehold 103, and detection region 104. A second oligonucleotide 110 contains a barcode and complementary regions 112, 113 to the displacement and detection regions of the first oligonucleotide, respectively.

FIG. 1C shows an example of a representative target C, e.g., an oligonucleotide (target oligonucleotide). This target includes complementary regions 123, 124 to the toehold and detection regions, respectively, in the first oligonucleotide of the detector molecule or a polynucleotide, protein, peptide, small molecule, or polynucleotide/protein complex 125.

FIG. 1D shows an example of a representative single stranded oligonucleotide D (helper strand) with complementary regions to the displacement and toehold regions of the first oligonucleotide in the detector molecule that displaces the second oligonucleotide from the detector molecule, specifically a displacement region 132 and a toehold region 133.

### EXAMPLES

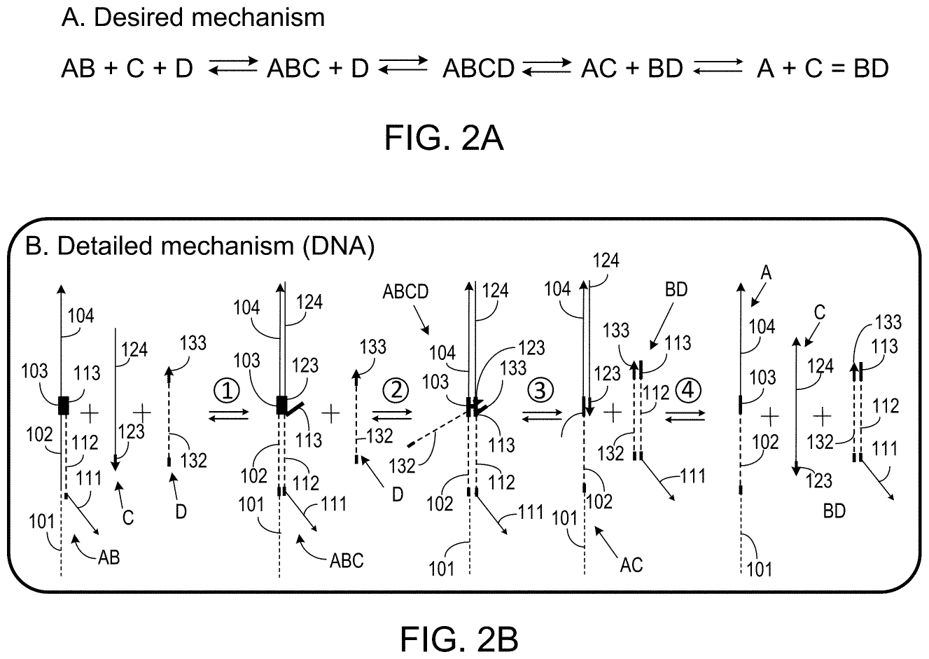

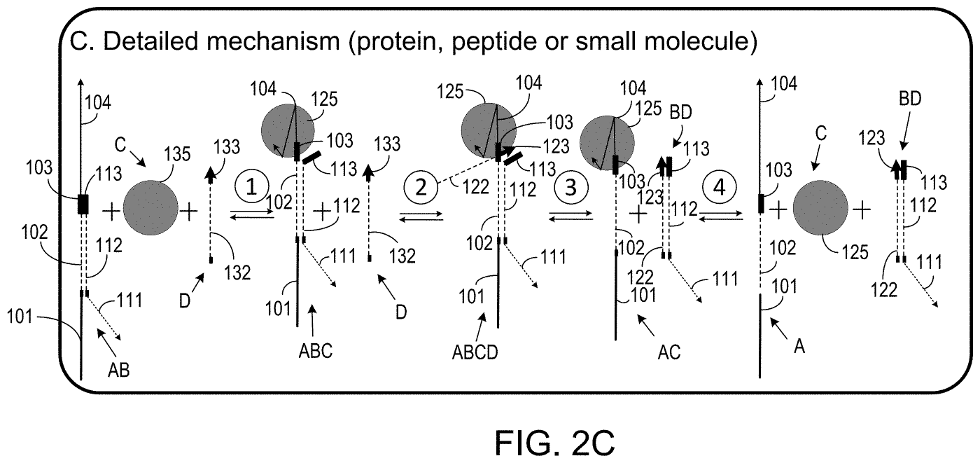

The invention is further described in the following examples, which do not limit the scope of the invention described in the claims. Referring to FIG. 2A, a formulaic example of the strand displacement processes described in EXAMPLE 1 and EXAMPLE 2 below involve four steps in which a detector molecule (AB) interacts with another molecule (D), in the presence of a target (C), to result in a molecule (A) and a detectable reporting molecular construct (BD).

### Example 1: Single Stranded Displacement with an Oligonucleotide Target

Referring to FIG. 2B, in this example, the target (target oligonucleotide) is an oligonucleotide with complementary regions to the detector and toehold regions in the first oligonucleotide in the detector molecule. The target oligonucleotide hybridizes to the toehold and detection regions in the detector molecule. The toehold region in the second oligonucleotide in the detector molecule becomes single stranded. The single stranded oligonucleotide (helper strand) including complementary regions to the toehold and displacement regions in the first oligonucleotide in the detector molecule hybridizes in the toehold region in the first oligonucleotide. The second oligonucleotide is displaced as the helper strand hybridizes with the displacement region in the first oligonucleotide.

### Example 2: Single Stranded Displacement with a Polynucleotide, Protein, Peptide, Small Molecule, or Polynucleotide/Protein Complex Target

In this example, the target is a polynucleotide, protein, peptide, small molecule, or polynucleotide/protein complex (FIG. 2C). The target binds to the detector region in the first oligonucleotide in the detector molecule. Binding of the target overlaps with the toehold region and weakens bonding between the first and second oligonucleotides in the toehold region. The toehold region in the second oligonucleotide in the detector molecule becomes single stranded. The single stranded oligonucleotide (helper strand) including complementary regions to the toehold and displacement regions in the first oligonucleotide in the detector molecule hybridizes in the toehold region in the first oligonucleotide. The second oligonucleotide is displaced as the helper strand hybridizes with the displacement region in the first oligonucleotide. The resulting oligonucleotide complex includes the second and fourth oligonucleotides.

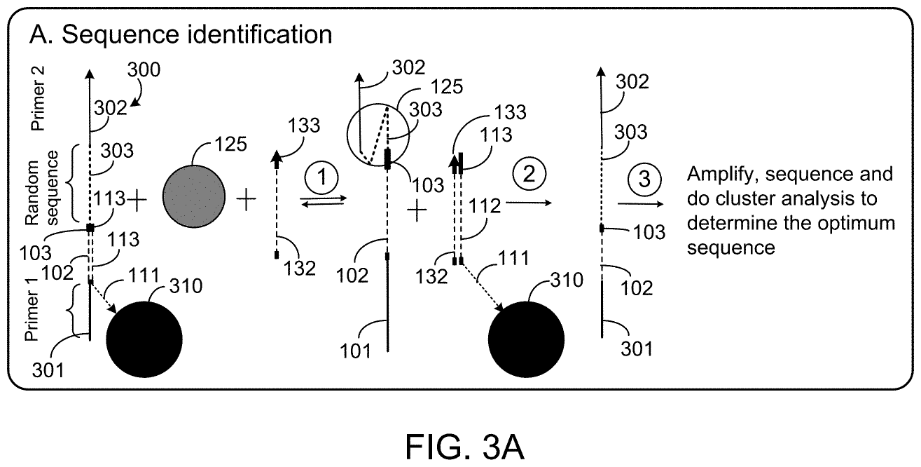

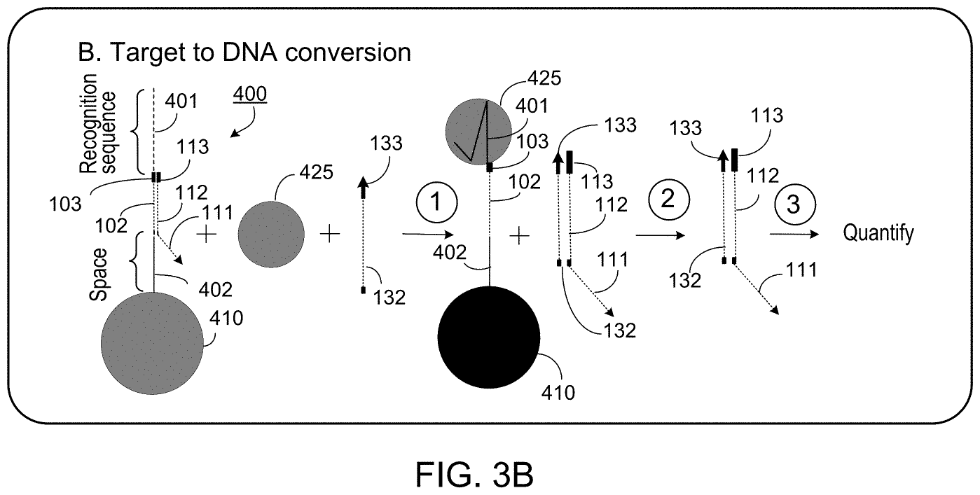

The detector molecule includes a substrate, e.g., a bead or a planar substrate, at the end with the spacer region in the first oligonucleotide (FIG. 3B). After binding of the target to the detector region and strand displacement, the first oligonucleotide and the target are removed from the solution. The remaining complex includes the second oligonucleotide from the detector molecule and the helper strand. The barcode is quantified to determine the concentration of the target.

### Example 3: Single Stranded Displacement to Design Detector Region Sequences

In this example, the detector molecule 300 includes a first primer 301 and second primer 302 one either end of the first oligonucleotide and a bead 310 attached at the bar code end 111 of the second oligonucleotide (FIG. 3A). The detector region 303 in the first oligonucleotide is a random sequence. Similar to what was shown previously in FIG. 2C, the target 125 binds to the detector molecule overlapping the toehold region 103. The toehold region 103 becomes single stranded and the displacement oligonucleotide displaces the second oligonucleotide (111, 112, 113, and 310) from the detector molecule. The resulting complex including the second oligonucleotide (111, 112, 113, and 310) and helper strand (132, 133) are removed from solution using the bead 310. The first oligonucleotide is amplified and sequenced to determine the sequenced of the detector region.

### Example 4: Diagnostic Uses

In this example, a biological sample is obtained from a subject and contacted with a detector molecule 400. The detector molecule includes a detector region 401 in the first oligonucleotide with affinity to a target 425 (FIG. 3B). The first oligonucleotide includes a spacer 402 that attaches the oligonucleotide to a bead 410. The target 425 can be a biomarker found in a blood sample, a urine sample, a biopsy sample, or a saliva sample. Upon contacting the detector molecule to the biological sample and under conditions where the target 425 will bind to the detector region 401, the second oligonucleotide (111, 112, and 113) is displaced. The concentration of the displaced strand is determined and thereby determining the concentration of the target in the sample.

Further analysis of the sample includes comparing the concentration of the target in the sample to the level of the target in a reference sample. Comparing the level of the target in the sample from a subject can be increased or higher than the reference sample, can be decreased or lower than the reference sample, or can be the same as the reference sample.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed:

1. A method for detecting a target in a sample, the method comprising:
contacting the sample with a detector molecule,
wherein the detector molecule comprises a first oligonucleotide and a second oligonucleotide, wherein the first oligonucleotide in order from 5′ to 3′ or 5′ to 3′ comprises a spacer region, a displacement region, a toehold region, and a detection region and the second oligonucleotide comprises complementary regions to the displacement and toehold regions of the first oligonucleotide, with a third oligonucleotide comprising regions complementary to the displacement and toehold regions in the first oligonucleotide, under conditions that allow binding of the third oligonucleotide to the first oligonucleotide at both the displacement and toehold regions of the first oligonucleotide, thereby displacing the second oligonucleotide; and
detecting the second oligonucleotide displaced from the first oligonucleotide.

2. The method of claim 1, wherein the first, second, and third oligonucleotides comprise DNA, RNA, non-natural nucleic acids, or a combination thereof.

3. The method of claim 2, wherein the first, second, and third oligonucleotides comprise DNA.

4. The method of claim 1, wherein detecting comprises determining the concentration of the displaced second oligonucleotide by PCR or sequencing.

5. The method of claim 1, wherein the detection region in the first oligonucleotide binds at least one target.

6. The method of claim 1, wherein the first oligonucleotide is further fixed to a substrate at one end of the oligonucleotide.

7. The method of claim 6, wherein the substrate is a bead or planar substrate.

8. The method of claim 7, wherein the substrate is a bead.

9. The method of claim 8, wherein the substrate is a planar substrate.

10. The method of claim 6, further comprises removing the detector molecule and contacting the sample with a second detector molecule against a second target.

11. The method of claim 1, wherein the second oligonucleotide is further fixed to a substrate at one end of the oligonucleotide.

12. The method of claim 11, wherein the substrate is a bead or planar substrate.

13. The method of claim 12, wherein the substrate is a bead.

14. The method of claim 12, wherein the substrate is a planar substrate.

15. The method of claim 1, wherein the target is a polypeptide or protein, or a combination thereof.

16. The method of claim 1, wherein the target is complexed with a polypeptide or polynucleotide.

17. The method of claim 1, wherein the sample is a biological sample, e.g., a blood sample, a urine sample, a biopsy sample, or a saliva sample.

18. The method of claim 1, further comprises the target binds to the detector region.

19. A method for identifying or producing a detector molecule for a target, a method comprising:
contacting a sample with the detector molecule,
wherein the sample comprises the target,
wherein the detector molecule comprises a first oligonucleotide and a second oligonucleotide, wherein the first oligonucleotide in order from 5′ to 3′ or 5′ to 3′ comprises a first primer, a displacement region, a toehold region, a detection region, and a second primer and the second oligonucleotide comprises complementary regions to the displacement and toehold regions of the first oligonucleotide and a barcode, with a third oligonucleotide comprising regions complementary to the displacement and toehold regions in the first oligonucleotide, under conditions that allow binding of the third oligonucleotide to the first oligonucleotide at both the displacement and toehold regions of the first oligonucleotide, thereby displacing the second oligonucleotide; and
determining the sequence of the detection region in the first oligonucleotide.

20. A composition for detecting a target in a sample, a composition comprising:
a detector molecule, wherein the detector molecule comprises a first oligonucleotide and a second oligonucleotide, wherein the first oligonucleotide in order from 5′ to 3′ or 5′ to 3′ comprises a spacer region, a displacement region, a toehold region, and a detection region and the second oligonucleotide comprises complementary regions to the displacement and toehold regions of the first oligonucleotide.
